(19) United States
(12) Patent Application Publication
(10) Pub. No.: US 2026/0317257 A1
(43) Pub. Date: Oct. 15, 2026

(54) Method for Preparing Hexafluoro-1,3-Butadiene

(71) Applicants: SINOCHEM LANTIAN ELECTRONIC MATERIALS (HANGZHOU) CO., LTD., Hangzhou, Zhejiang (CN); ZHEJIANG RESEARCH INSTITUTE OF CHEMICAL INDUSTRY CO., LTD, Hangzhou, Zhejiang (CN); SINOCHEM LANTIAN CO., LTD., Hangzhou, Zhejiang (CN)

(72) Inventors: Wucan LIU, Hangzhou, Zhejiang (CN); Haifeng WU, Hangzhou, Zhejiang (CN); Hao CHENG, Hangzhou, Zhejiang (CN); Jingtian ZHANG, Hangzhou, Zhejiang (CN)

(73) Assignees: SINOCHEM LANTIAN ELECTRONIC MATERIALS (HANGZHOU) CO., LTD., Hangzhou, Zhejiang (CN); ZHEJIANG RESEARCH INSTITUTE OF CHEMICAL INDUSTRY CO., LTD, Hangzhou, Zhejiang (CN); SINOCHEM LANTIAN CO., LTD., Hangzhou, Zhejiang (CN)

(21) Appl. No.: 18/877,485

(22) PCT Filed: Nov. 15, 2024
(86) PCT No.: PCT/CN2024/132459

§ 371 Date: Dec. 20, 2024

(30) Foreign Application Priority Data
Dec. 1, 2023 (CN) 202311639961.2

(51) Int. Cl. C07C 17/281; C07C 17/383

(52) U.S. Cl. CPC C07C 17/281; C07C 17/383

(57) ABSTRACT

Provided is method for preparing hexafluoro-1,3-butadiene. The method includes: step (1), a trifluoroethenyl zinc bromide solution, a catalyst, and a first organic solvent are placed into a pressure resistant reactor, bromotrifluoroethylene is added after displacement with an inert gas, and hexafluoro-1,3-butadiene is obtained through reaction; and step (2), after the reaction is completed, a gas-phase material is allowed to pass through a circulating spray system filled with a second organic solvent to absorb excess bromotrifluoroethylene and fluoro-olefin impurities, and the unabsorbed crude hexafluoro-1,3-butadiene is purified through distillation, where the second organic solvent is selected from the polar aprotic organic solvent or ionic liquid. In the present disclosure, the method for preparing hexafluoro-1,3-butadiene through cross-coupling of bromotrifluoroethylene and trifluoroethenyl zinc bromide is realized.

This application claims priority to Chinese Patent Application No. 202311639961.2 filed to the China National Intellectual Property Administration on Dec. 1, 2023, the disclosure of which is hereby incorporated by reference in its entirety.

### TECHNICAL FIELD

The present disclosure relates fluorine-containing electronic gases, and in particular, to method for preparing hexafluoro-1,3-butadiene.

### BACKGROUND

Hexafluoro-1,3-butadiene (C₄F₆) is a colorless liquefiable fluorine-containing gas, with a boiling point of 5.5° C. at atmospheric pressure and a liquid density of 1.44 g/mL at 15° C. Currently, the hexafluoro-1,3-butadiene is mainly used as a dry etching gas for precision etching (accuracy up to 100 nm) of critical dimensions for the production of a rectification circuit board containing Cu and with a low K dielectric constant, and has better selectivity and depth-to-width ratio than other etching gases. For example, compared with octafluorocyclobutane (c-C₄F₈), an etching depth-to-width ratio of the C₄F₆ may reach 10, and c-C₄F₈ is only 3, such that the C₄F₆ is suitable for an extremely narrow line width process. The C₄F₆ only etches a silicon oxide film without affecting a photoresist, a silicon film, and a nitride film, and has excellent etching selectivity. Furthermore, the hexafluoro-1,3-butadiene also has good environment performance with ODP=0 and GWP (100)=290 and an atmospheric presence time of only 1.9 d, and thus a green and environmentally-friendly etching gas with an extremely-low greenhouse effect. Therefore, with the development of the integrated circuit industry and concerns about greenhouse gases, the hexafluoro-1,3-butadiene having an optimal etching effect and environmentally-friendly features is bound to become a dominant product and widely used in the laser etchant market.

The preparation and purification of the hexafluoro-1,3-butadiene is one of the hotspots of research in recent years, the preparation of the hexafluoro-1,3-butadienein the related art is mainly reported as follows.

WO 2006/026400 disclosed that chlorotrifluoroethylene (CF₂═CFCl) was used as a raw material, bromotrifluoroethylene (CF₂═CFBr) was prepared through hydrodechlorination, bromination, dehydrobromination, then trifluoroethenyl zinc bromide (CF₂═CFZnBr) was prepared through a reaction with zinc powder, and finally a homocoupling reaction was performed under the presence of Fe³⁺ or Cu²⁺ ions, so as to obtain hexafluoro-1,3-butadienein. Although materials used in the method are low in cost, various dangerous production processes are involved, steps are relatively lone, and an intermediate product trifluoroethylene has the risk of self-polymerization and is not suitable for industrialized scale-up production.

CN 104829415 disclosed a process route of using tetrafluoroethane (HFC-134a) as a raw material to prepare the hexafluoro-1,3-butadienein. First, the tetrafluoroethane and bromine react at a high temperature to obtain 1,1-dibromotetrafluoroethane; then a reaction with a zinc powder is performed in a polar aprotic solvent to prepare trifluoroethenyl zinc bromide; and finally, homocoupling is performed under a Fe³⁺ oxidant to obtain the hexafluoro-1,3-butadienein, and a total yield of the reaction is 47%. The reaction uses a cheap refrigerant HFC-134a as a raw material, and has a relatively short process route. However, the activity of the 1,1-dibromotetrafluoroethane is low, and the yield when the trifluoroethenyl zinc bromide is prepared with the zinc powder is high, causing the process route to be high in actual unit consumption, large in the amount of three wastes, and low in actual industrialization prospect.

US304630 disclosed a method for preparing hexafluoro-1,3-butadiene using chlorotrifluoroethylene as a raw material. The chlorotrifluoroethylene first reacts with Iodine in 35-40 Chloride (ICI) a sealing system, so as to obtain 1,2-dichloro-1,2,2-trifluoroiodoethane, and then is coupled to equivalent mercury under ultraviolet lamp irradiation, so as to obtain 1,2,3,4-tetrachloro-1, 1,2,3,4,4-hexafluorobutane; and finally, dechlorination is performed on the 1,2,3,4-tetrachloro-1,1,2,3,4,4-hexafluorobutane in an alcohol solvent under the action of the zinc powder, so as to obtain the hexafluoro-1,3-butadiene. In this method, chemical equivalent amounts of iodine chloride and mercury are required to participate the reaction, mercury iodide is one of the products and has large toxicity, and reagents used are expensive.

US2894043 disclosed that dimerization was performed on 1,2-dichloro-difluoroethene (CFCl═CFCl) under the presence of a fluorine gas to synthetize an intermediate product 1,2,3,4-tetrachlorohexafluorobutane, and then dechlorination is performed by using zinc powder, so as to obtain a target product hexafluoro-1,3-butadiene. In this method, at the fluorination dimerization stage, a low temperature condition (−70° C.) is required, and a high risk gas F₂ is required as well. U.S. Pat. No. 2,676,193 improved this method, but the reaction still needed high temperature (300° C.) and high pressure (12 MPa) conditions, and a reaction yield was lower, which was only 30-40%, and product separation was difficult due to many by-products.

WO 2018235883 disclosed that chlorotrifluoroethylene was used as a raw material, and a homocoupling reaction was catalyzed under the presence of a palladium catalyst, a phosphorus ligand, and zinc powder, so as to obtain hexafluoro-1,3-butadiene, with a highest yield being 86.1%. Patent CN 116693365 disclosed that chlorotrifluoroethylene and trifluoroethylene were used as raw materials, and a cross-coupling reaction was performed under the presence of an active palladium catalyst and a basic compound, so as to obtain hexafluoro-1,3-butadiene, with a highest reaction yield being 83%. Although the method is simple in process, expensive palladium is used as the catalyst, such that the method does not offer advantages in terms of raw material costs.

The purification of the hexafluoro-1,3-butadiene in the related art is mainly reported as follows.

US6544319 disclosed a method for purifying hexafluoro-1,3-butadiene using adsorption. The method is low in cost and mild in condition, and may effectively adsorb azeotropic fluorinated olefin compound and alcohol impurities. However, the method releases heat during the adsorption of the alcohol impurities, rearrangement isomerization is easy to occur when the hexafluoro-1,3-butadiene is heated, thereby generating impurities such as hexafluoro-2-butyne difficult to remove.

CN 111247120 disclosed that distillation purification was performed on crude hexafluoro-1,3-butadiene containing octafluoro-1-butene, octafluoro-2-butene, heptafluoro-1-butene, and heptafluoro-2-butene by using oxygenated hydrocarbons such as alcohols, ketones, ethers, etc., halogenated saturated hydrocarbons, halogenated unsaturated hydrocarbons, etc. as an extractive solvent, and a theoretical plate was 14, and a hexafluoro-1,3-butadiene product with purity being 99.99% was obtained.

CN 111138240 disclosed a method for removing moisture in hexafluoro-1,3-butadiene by using dioctyl phthalate, ethylene glycol, dimethyl diamide, and glycerol as an extractant. A moisture content of the purified product taken is 10-12 ppm. However, that the moisture in the product is removed by using the extractant is disclosed only, and the separated crude hexafluoro-1,3-butadiene does not contain heptafluorobutene impurities. The purity of the hexafluoro-1,3-butadiene product is not disclosed.

CN 116903438 disclosed the purification of hexafluorobutene by using an extraction rectification and adsorption combined technology. Furfural, methyl ethyl ketone, and cyclopentanone are used as extractants, organic base and copper salt doped modified graphene oxide are used as adsorbents, and the highest product purity may reach more than 5 N, but the purity of the raw materials is not given.

In views of deficiencies in the related art, a method for preparing hexafluoro-1,3-butadiene with a simple process and low cost is required.

### SUMMARY

In order to improve the problems of high costs, more steps, poor yields, safety hazards, and the like in a method for preparing hexafluoro-1,3-butadiene in the related art, the present disclosure provides a method for preparing hexafluoro-1,3-butadiene, so as to make up deficiencies in existing methods for preparing hexafluoro-1,3-butadiene. A technical route of the present disclosure is as follows:

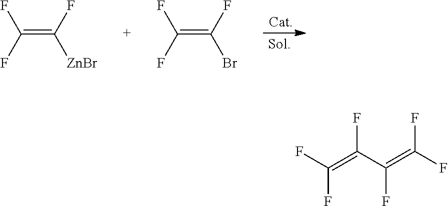

In order to implement the objective of the present disclosure, the present disclosure uses the following technical solutions.

A method for preparing hexafluoro-1,3-butadiene includes the following steps.

At step (1), a trifluoroethenyl zinc bromide solution, a catalyst, and a first organic solvent are placed into a pressure resistant reactor, bromotrifluoroethylene is added after displacement with an inert gas, and hexafluoro-1,3-butadiene is obtained through reaction.

The catalyst includes a monovalent copper salt or a monovalent copper oxide and an organophosphine ligand.

Preferably, the monovalent copper salt is selected from any one or more of cuprous iodide, cuprous bromide, cuprous chloride, and cuprous acetate, and the monovalent copper oxide is cuprous oxide.

The organophosphine ligand is selected from any one or more of 1,10-phenanthroline, PPh₃, PR₁R₂R₃, PPhR₁R₂, PPh₂R₁, 1,2-bis(diphenylphosphino) ethane, and 2,2′-bis(diphenylphosphino) biphenyl. R₁, R₂, and R₅ are each independently selected from any one of n-propyl, isopropyl, n-butyl, isobutyl, and tert-butyl. Preferably, the organophosphine ligand is selected from one of the 1,10-phenanthroline, the PPh₃, the 1,2-bis(diphenylphosphino) ethane, and the 2,2′-bis(diphenylphosphino) biphenyl.

The first organic solvent uses a polar aprotic organic solvent.

The first organic solvent is selected from any one or more of N,N-dimethylformamide, N,N-dimethylacetamide, hexamethylphosphoramide, dimethyl sulfoxide, sulfolane, and N-methyl-2-pyrrolidone, and a moisture content of the polar aprotic organic solvent used in the first organic solvent is ≤500 ppm.

Preferably, the organic solvent in step (1) is selected from one of the N,N-dimethylformamide and N,N-dimethylacetamide, with the moisture content≤200 ppm.

At step (2), after the reaction is completed, a gas-phase material is allowed to pass through a circulating spray system filled with a second organic solvent to absorb excess bromotrifluoroethylene and fluoro-olefin impurities generated during reaction, and the unabsorbed crude hexafluoro-1,3-butadiene is purified through distillation.

The second organic solvent is selected from the polar aprotic organic solvent or ionic liquid.

The polar aprotic organic solvent used in the second organic solvent is selected from any one or more of the N,N-dimethylformamide, the N,N-dimethylacetamide, the hexamethylphosphoramide, the dimethyl sulfoxide, and the sulfolane; and the ionic liquid is selected from any one or more of 1-butyl-3-methylimidazole phosphate, 1-butyl-3-methylimidazolium methanesulfonate, 1-butyl-3-methylimidazolium acetate, 1-butyl-3-methylimidazolium trifluoroacetate, 1-butyl-3-methylimidazolium nitrate, 1-butyl-3-methylimidazolium (L)-lactate, and 1-butyl-3-methylimidazolium hexafluorophosphate.

Preferably, the second organic solvent is selected from one of the N,N-dimethylformamide, the N,N-dimethylacetamide, the 1-butyl-3-methylimidazolium trifluoroacetate, the 1-butyl-3-methylimidazolium nitrate, and the 1-butyl-3-methylimidazolium hexafluorophosphate.

A mole ratio of trifluoroethenyl zinc bromide in the trifluoroethenyl zinc bromide solution to the monovalent copper salt or monovalent copper oxide is 1:(0.01-0.3); and a mole ratio of the monovalent copper salt or monovalent copper oxide to the organophosphine ligand is 1:(1.0-10).

Preferably, the mole ratio of the trifluoroethenyl zinc bromide in the trifluoroethenyl zinc bromide solution to the monovalent copper salt or monovalent copper oxide is 1:(0.01-0.20); and the mole ratio of the monovalent copper salt or monovalent copper oxide to the organophosphine ligand is 1:(1.0-5.0).

Further preferably, the mole ratio of the trifluoroethenyl zinc bromide in the trifluoroethenyl zinc bromide solution to the monovalent copper salt or monovalent copper oxide is 1:(0.05-0.10); and the mole ratio of the monovalent copper salt or monovalent copper oxide to the organophosphine ligand is 1:(1.0-3.0).

A mole ratio of the trifluoroethenyl zinc bromide in the trifluoroethenyl zinc bromide solution in step (1) to the bromotrifluoroethylene is 1:(1.0-10.0).

Preferably, the mole ratio of the trifluoroethenyl zinc bromide in the trifluoroethenyl zinc bromide solution in step (1) to the bromotrifluoroethylene is 1:(1.0-5.0).

A reaction temperature in step (1) is 60-140° C., a reaction pressure is 0.05-2.0 MPa, and a reaction time is 3-24 h.

Preferably, the reaction temperature in step (1) is 80-120° C., the reaction pressure is 0.3-1.0 MPa, and the reaction time is 6-12 h.

The circulating spray system in step (2) is a two-stage circulating spray absorption system, with a temperature being −10 to 20° C. and a pressure being 0-0.5 MPa.

Preferably, the circulating spray system in step (2) is the two-stage circulating spray absorption system, with the temperature being −5 to 10° C. and the pressure being 0-0.2 MPa.

In the method for preparing hexafluoro-1,3-butadiene, an absorbed organic solution in the circulating spray system in step (2) is treated through distillation, and the bromotrifluoroethylene is recycled and directly circulated for use.

A content of the bromotrifluoroethylene in the crude hexafluoro-1,3-butadiene treated by the circulating spray system is ≤0.3%, a content of heptafluorobutene is ≤0.02%, and a hexafluoro-1,3-butadiene product with purity ≥99.99% may be obtained after distillation.

The present disclosure has the following beneficial effects.

(1) In the present disclosure, the cheap monovalent copper salt or oxide is used as the catalyst, and the organophosphorus as the ligand, such that the method for preparing the hexafluoro-1,3-butadiene through the cross-coupling of the bromotrifluoroethylene and the trifluoroethenyl zinc bromide is realized. Compared with an oxidative homocoupling process, the method has the advantages of being low in raw material cost, less in discharge amount of the three wastes, etc.

(2) The reaction product passes through a spray circulation absorption apparatus filled with a special solvent, excess bromotrifluoroethylene and the difficultly-separated impurity heptafluorobutene may be dissolved in the solvent, the content of the bromotrifluoroethylene in the obtained crude hexafluoro-1,3-butadiene is ≤0.3%, and the content of the heptafluorobutene is ≤0.02%, such that the pressure for the distillation and purification of subsequent products is reduced. Through conventional distillation, the product with the purity ≥99.99% may be obtained.

(3) In the present disclosure, through simple distillation, circulated use of the bromotrifluoroethylene is realized, thereby improving a utilization rate of the trifluoroethylene.

### BRIEF DESCRIPTION OF THE DRAWINGS

FIG. 1 shows an apparatus for preparing hexafluoro-1,3-butadiene according to embodiments of the present disclosure. The apparatus includes a continuous preparation apparatus for a trifluoroethenyl zinc bromide solution in a method for continuously preparing hexafluoro-1,3-butadiene, including: 1: bromotrifluoroethylene steel cylinder; 2: trifluoroethenyl zinc bromide solution steel cylinder; 3: pressure resistant reactor; 4: condenser; 5: buffer tank; 6: first spray circulation absorption tower; 7: first spray circulation absorption tower; and 8: coarse product tank.

FIG. 2 is a chromatographic analysis diagram of a product after distillation in Embodiment 10.

### DETAILED DESCRIPTION OF THE EMBODIMENTS

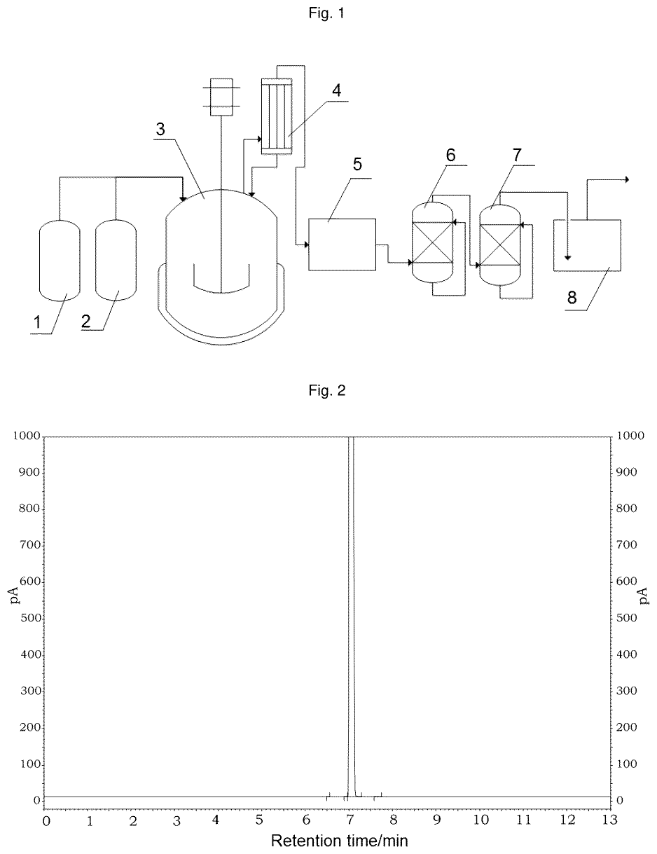

Referring to FIG. 1, a preparation system used in the present disclosure is described first. Pressure resistant reactor 3, condenser 4, buffer tank 5, first spray circulation absorption tower 6, second spray circulation absorption tower 7, and coarse product tank 8 are included.

Bromotrifluoroethylene steel cylinder 1 and trifluoroethenyl zinc bromide solution steel cylinder 2 are connected to a feeding port of the pressure resistant reactor 3, a top discharging port of the pressure resistant reactor 3 is connected to a feeding port of the condenser 4, a discharging port of the condenser 4 is connected to a feeding port of the buffer tank 5, a discharging port of the buffer tank 5 is connected to a feeding port of the first spray circulation absorption tower 6, a discharging port of the first spray circulation absorption tower 6 is connected to a feeding port of the second spray circulation absorption tower 7, a discharging port of the second spray circulation absorption tower 7 is connected to the coarse product tank 8, and a discharging port of the coarse product tank 8 is connected to a distillation device.

An implementation test is performed on the above system based on the following reasons.

### Embodiment 1

(1) 1810 g of a N,N-dimethylacetamide solution (a mass fraction of 25%, 2.0 mol) of trifluoroethenyl zinc bromide, 1000 g of N,N-dimethylacetamide (moisture being 150 ppm), 38.2 g of cuprous iodide (0.2 mol), and 72.2 g of 1,10-phenanthroline (0.4 mol) were added to a 5 L pressure resistant reactor made of 316L provided with a condensation reflux apparatus. The reactor was replaced with high purity nitrogen, and pumped to a vacuum of ≥0.1 MPa. 644 g of bromotrifluoroethylene (4.0 mol) was added to the pressure resistant reactor, stirring started, a temperature in the reactor was heated to 100° C., a temperature of a jacket medium of a condenser was 20° C., a pressure of the reactor was 0.8 MPa, after 6 h of a reaction, a sample was taken from a liquid phase tube of the pressure resistant reactor to perform analysis through a nuclear magnetic fluorine spectrometry internal standard method (benzotrifluoride being an internal standard substance), and the trifluoroethenyl zinc bromide had been completely converted.

(2) In two-stage circulating spray apparatuses with a capacity of 1 L each, 1 L of 1-butyl-3-methylimidazolium hexafluorophosphate was added, an inner temperature was controlled at 4-6° C., and an internal circulating pump was turned on, with a flow rate being 100 mL/min. A gas phase outlet regulating valve of the pressure resistant reactor was turned on, the reactor was heated to 130° C., a gas phase product entered the two-stage circulating spray apparatus, and unabsorbed exhaust gases were collected in a cold trap absorption apparatus using liquid nitrogen as cooling liquid. Results showed that, 244.8 g of crude hexafluoro-1,3-butadiene was collected in a cold trap 1, with purity being 97.82%, converted to 239.4 g on a hundredweight basis, a theoretical yield was 324.0 g, and a yield was 73.9%. Contents of various impurities in the crude hexafluoro-1,3-butadiene were shown in Table 1.

(3) The 1-butyl-3-methylimidazolium hexafluorophosphate solution in the two-stage circulating spray apparatus was added to the pressure resistant reactor for distillation, the temperature was heated to 120° C., and dissolved fluoroolefin was evaporated and collected in the cold trap absorption apparatus using the liquid nitrogen as the cooling liquid. Results showed that, a total of 341.9 g was collected in a cold trap 2, a chromatographic content of the bromotrifluoroethylene was 89.50%, a chromatographic content of the hexafluoro-1,3-butadiene was 9.75%, and contents of other components were shown in Table 2.

### Embodiment 2

Operations in this embodiment were the same as Embodiment 1, and a difference between this embodiment and Embodiment 1 only lied in that, the adding of 38.2 g of the cuprous iodide (0.2 mol) in the 5 L pressure resistant reactor made of 316L was replaced with the adding of 19.1 g of the cuprous iodide (0.1 mol), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and a conversion rate of trifluoroethenyl zinc bromide was 94.5%.

234.2 g of the crude hexafluoro-1,3-butadiene in step (2) was collected a in cold trap 1, with purity being 96.60%, converted to 226.2 g on a hundredweight basis, a theoretical yield was 324.0 g, and a yield was 69.8%. Contents of various impurities in the crude hexafluoro-1,3-butadiene were shown in Table 1.

A total of 358.7 g was collected in the cold trap 2 in step (3), a chromatographic content of the bromotrifluoroethylene was 90.80%, a chromatographic content of the hexafluoro-1,3-butadiene was 8.55%, and contents of other components were shown in Table 2.

### Embodiment 3

Operations in this embodiment were the same as Embodiment 1, and a difference between this embodiment and Embodiment 1 only lied in that, the adding of 38.2 g of cuprous iodide (0.2 mol) in the 5 L pressure resistant reactor made of 316L was replaced with the adding of 19.8 g of cuprous chloride (0.2 mol), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and trifluoroethenyl zinc bromide had been completely converted.

244.0 g of the crude hexafluoro-1,3-butadiene in step (2) was collected a in cold trap 1, with purity being 96.32% and 235 g on a hundredweight basis, a theoretical yield was 324.0 g, and a yield was 72.5%. Contents of various impurities in the crude hexafluoro-1,3-butadiene were shown in Table 1.

A total of 339.3 g was collected in the cold trap 2 in step (3), a chromatographic content of the bromotrifluoroethylene was 89.25%, a chromatographic content of the hexafluoro-1,3-butadiene was 10.10%, and contents of other components were shown in Table 2.

### Embodiment 4

Operations in this embodiment were the same as Embodiment 1, and a difference between this embodiment and Embodiment 1 only lied in that, the adding of 72.2 g of 1,10-phenanthroline (0.4 mol) in the 5 L pressure resistant reactor made of 316L was replaced with the adding of 104.9 g of triphenylphosphine (0.4 mol), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and a conversion rate of trifluoroethenyl zinc bromide was 97.9%.

254.0 g of the crude hexafluoro-1,3-butadiene in step (2) was collected a in cold trap 1, with purity being 95.83% and 243.4 g on a hundredweight basis, a theoretical yield was 324.0 g, and a yield was 75.1%. Contents of various impurities in the crude hexafluoro-1,3-butadiene were shown in Table 1.

A total of 340.6 g was collected in the cold trap 2 in step (3), a chromatographic content of the bromotrifluoroethylene was 91.40%, a chromatographic content of the hexafluoro-1,3-butadiene was 8.29%, and contents of other components were shown in Table 2.

### Embodiment 5

Operations in this embodiment were the same as Embodiment 1, and differences between this embodiment and Embodiment 1 only lied in that, the adding of 1810 g of the N, N-dimethylacetamide solution (the mass fraction of 25%, 2.0 mol) of the trifluoroethenyl zinc bromide in the 5 L pressure resistant reactor made of 316L was replaced with the adding of 1810 g of a N, N-dimethylformamide solution (a mass fraction of 25%, 2.0 mol) of trifluoroethenyl zinc bromide, and 1000 g of the N,N-dimethylacetamide (moisture being 150 ppm) was replaced with 1000 g of the N,N-dimethylformamide (moisture being 150 ppm), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and trifluoroethenyl zinc bromide had been completely converted.

251.0 g of the crude hexafluoro-1,3-butadiene in step (2) was collected a in cold trap 1, with purity being 96.59% and 242.4 g on a hundredweight basis, a theoretical yield was 324.0 g, and a yield was 74.8%. Contents of various impurities in the crude hexafluoro-1,3-butadiene were shown in Table 1.

A total of 338.7 g was collected in the cold trap 2 in step (3), a chromatographic content of the bromotrifluoroethylene was 89.85%, a chromatographic content of the hexafluoro-1,3-butadiene was 9.31%, and contents of other components were shown in Table 2.

### Embodiment 6

Operations in this embodiment were the same as Embodiment 1, and a difference between this embodiment and Embodiment 1 only lied in that, the temperature in the reactor being heated to 100° C. was replaced with the temperature in the reactor being heated to 80° C., with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and a conversion rate of trifluoroethenyl zinc bromide was 80.5%.

205.6 g of the crude hexafluoro-1,3-butadiene in step (2) was collected a in cold trap 1, with purity being 95.50% and 196.4 g on a hundredweight basis, a theoretical yield was 324.0 g, and a yield was 60.6%. Contents of various impurities in the crude hexafluoro-1,3-butadiene were shown in Table 1.

A total of 391.28 g was collected in the cold trap 2 in step (3), a chromatographic content of the bromotrifluoroethylene was 93.31%, a chromatographic content of the hexafluoro-1,3-butadiene was 5.75%, and contents of other components were shown in Table 2.

### Embodiment 7

Operations in this embodiment were the same as Embodiment 1, and a difference between this embodiment and Embodiment 1 only lied in that, the second organic solvent 1-butyl-3-methylimidazolium hexafluorophosphate in step (2) was replaced with 1-butyl-3-methylimidazolium trifluoroacetate, with other conditions unchanged. Results were shown as follows.

253.0 g of the crude hexafluoro-1,3-butadiene in step (2) was collected a in cold trap 1, with purity being 96.20% and 247.2 g on a hundredweight basis, a theoretical yield was 324.0 g, and a yield was 76.3%. Contents of various impurities in the crude hexafluoro-1,3-butadiene were shown in Table 1.

A total of 337.85 g was collected in the cold trap 2 in step (3), a chromatographic content of the bromotrifluoroethylene was 89.61%, a chromatographic content of the hexafluoro-1,3-butadiene was 9.20%, and contents of other components were shown in Table 2.

### Embodiment 8

Operations in this embodiment were the same as Embodiment 1, and a difference between this embodiment and Embodiment 1 only lied in that, the temperature in the two-stage circulating spray apparatus in step (2) being controlled at 4-6° C. was replaced with the inner temperature of 2-4° C., with other conditions unchanged. Results were shown as follows.

233.8 g of the crude hexafluoro-1,3-butadiene in step (2) was collected a in cold trap 1, with purity being 97.85% and 228.8 g on a hundredweight basis, a theoretical yield was 324.0 g, and a yield was 70.6%. Contents of various impurities in the crude hexafluoro-1,3-butadiene were shown in Table 1.

A total of 356.4 g was collected in the cold trap 2 in step (3), a chromatographic content of the bromotrifluoroethylene was 88.02%, a chromatographic content of the hexafluoro-1,3-butadiene was 11.46%, and contents of other components were shown in Table 2.

### Embodiment 9

Operations in this embodiment were the same as Embodiment 1, and a difference between this embodiment and Embodiment 1 only lied in that, the bromotrifluoroethylene recycled in step (1) was used as a raw material, the bromotrifluoroethylene purity of the recycled bromotrifluoroethylene was 93.20%, and the hexafluoro-1,3-butadiene was 6.25%.

A feeding amount was 691 g (bromotrifluoroethylene converted to 644 g on a hundredweight basis), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and trifluoroethenyl zinc bromide had been completely converted.

330.6 g of the crude hexafluoro-1,3-butadiene in step (2) was collected a in cold trap 1, with purity being 96.33%, converted to 318.4 g on a hundredweight basis, a theoretical yield was 324.0 g, and a yield was 98.30%. Contents of various impurities in the crude hexafluoro-1,3-butadiene were shown in Table 1.

A total of 341.9 g was collected in the cold trap 2 in step (3), a chromatographic content of the bromotrifluoroethylene was 88.90%, a chromatographic content of the hexafluoro-1,3-butadiene was 10.52%, and contents of other components were shown in Table 2.

TABLE 1
Table of contents of components in crude hexafluoro-1,3-
butadiene in each batch

| | | | | | C₄F₆ | |
|---|---|---|---|---|---|---|
| | C₄F₆/ | C₂F₃H/ | C₂F₃Br/ | C₄F₇H/ | Dimer/ | |
| Batch | % | % | % | % | % | Other |
| Embodi- | 97.82 | 1.9451 | 0.1720 | 0.0128 | 0.0051 | 0.0450 |
| ment 1 | | | | | | |
| Embodi- | 96.60 | 3.0794 | 0.2190 | 0.0095 | 0.0041 | 0.0880 |
| ment 2 | | | | | | |
| Embodi- | 96.32 | 3.3414 | 0.2440 | 0.0173 | 0.0077 | 0.0696 |
| ment 3 | | | | | | |
| Embodi- | 95.83 | 3.8767 | 0.2120 | 0.0172 | 0.0089 | 0.0552 |
| ment 4 | | | | | | |
| Embodi- | 96.59 | 3.1879 | 0.1598 | 0.0135 | 0.0048 | 0.0440 |
| ment 5 | | | | | | |
| Embodi- | 95.50 | 4.1466 | 0.2848 | 0.0139 | 0.0059 | 0.0488 |
| ment 6 | | | | | | |
| Embodi- | 96.20 | 3.6007 | 0.1075 | 0.0062 | 0.0060 | 0.0796 |
| ment 7 | | | | | | |
| Embodi- | 97.85 | 1.9743 | 0.1143 | 0.0105 | 0.0038 | 0.0471 |
| ment 8 | | | | | | |
| Embodi- | 96.33 | 3.2966 | 0.2698 | 0.0182 | 0.0063 | 0.0791 |
| ment 9 | | | | | | |

TABLE 2
Table of contents of components in absorption liquid in
each batch

| | | | | | C₄F₆ | |
|---|---|---|---|---|---|---|
| | C₄F₆/ | C₂F₃H/ | C₂F₃Br/ | C₄F₇H/ | Dimer/ | |
| Batch | % | % | % | % | % | Other |
| Embodi- | 9.75 | 0.5348 | 89.50 | 0.1252 | 0.0580 | 0.0320 |
| ment 1 | | | | | | |
| Embodi- | 8.55 | 0.4334 | 90.80 | 0.1188 | 0.0479 | 0.0499 |
| ment 2 | | | | | | |
| Embodi- | 10.10 | 0.3668 | 89.25 | 0.2176 | 0.0376 | 0.0280 |
| ment 3 | | | | | | |
| Embodi- | 8.29 | 0.1556 | 91.40 | 0.0892 | 0.0352 | 0.0300 |
| ment 4 | | | | | | |
| Embodi- | 9.31 | 0.5555 | 89.85 | 0.1440 | 0.0655 | 0.0750 |
| ment 5 | | | | | | |
| Embodi- | 5.75 | 0.7637 | 93.31 | 0.1060 | 0.0290 | 0.0413 |
| ment 6 | | | | | | |
| Embodi- | 9.20 | 0.5087 | 89.61 | 0.6200 | 0.0235 | 0.0378 |
| ment 7 | | | | | | |
| Embodi- | 11.46 | 0.2530 | 88.02 | 0.1443 | 0.0699 | 0.0528 |
| ment 8 | | | | | | |
| Embodi- | 10.52 | 0.1621 | 88.90 | 0.2754 | 0.0684 | 0.0741 |
| ment 9 | | | | | | |

Note: the second organic solvent is deducted.

### Embodiment 10

The crude hexafluoro-1,3-butadiene prepared in Embodiments 1-7 was used as the raw material for distillation experiments, and distillation tower parameters and distillation parameters were shown in Tables 3 and 4 below.

TABLE 3
Distillation tower parameter

| Tower | Inner | | | Number of |
|---|---|---|---|---|
| reactor | diameter of | Tower | | theoretical |
| volume | a tower | height | Filler | plates |
| 100 mL | 20 mm | 3.5 m | 316 L delta twist | 140 |

TABLE 4
Distillation process parameter

| External | | | |
|---|---|---|---|
| temperature of a | Tower reactor | Medium temperature | |
| tower reactor | pressure | of a condenser | Reflux ratio |
| 50 °C | 0.1-0.3 MPa | 0-5 °C | 5:1-10:1 |

A distillation feed was 800 g, a fraction before collection was 57.5 g, a product was 608 g, and reactor residues were 118.5 g. The purity of the product was 99.990%, a single batch distillation yield was 76.0%, and a material balance rate was 98.0%. A chromatographic analysis diagram of the product after distillation was shown in FIG. 2, and results were shown as follows.

| Retention time/min | Peak area/% | Substance name |
|---|---|---|
| 6.527 | 0.0005 | Trifluoroethylene |
| 6.925 | 0.0013 | Bromotrifluoroethylene |
| 7.008 | 99.9916 | Hexafluoro-1,3-butadiene |
| 7.659 | 0.0066 | Heptafluorobutene |
| Total | 100.0000 | |

### Comparative Embodiment 1

Operations in this embodiment were the same as Embodiment 1, and a difference between this embodiment and Embodiment 1 only lied in that, a monovalent copper salt catalyst was not added in step (1), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and a conversion rate trifluoroethenyl zinc bromide was 0.5%.

A hexafluoro-1,3-butadiene product was not collected in the cold trap in step (2).

### Comparative Embodiment 2

Operations in this embodiment were the same as Embodiment 1, and a difference between this embodiment and Embodiment 1 only lied in that, an organophosphine ligand was not added in step (1), with other conditions unchanged. Results were shown as follows.

After 6 h of the reaction in step (1), a sample was taken from a liquid phase tube of the pressure resistant reactor to perform analysis through a nuclear magnetic fluorine spectrometry internal standard method (benzotrifluoride being an internal standard substance), and a conversion rate trifluoroethenyl zinc bromide was 62.2%.

65.3 g of the crude hexafluoro-1,3-butadiene in step (2) was collected a in cold trap 1, with purity being 95.47%, converted to 62.3 g on a hundredweight basis, a theoretical yield was 162.0 g, and a yield was 38.5%.

A total of 444.4 g was collected in the cold trap 2 in step (3), a chromatographic content of the bromotrifluoroethylene was 92.81%, and a chromatographic content of the hexafluoro-1,3-butadiene was 6.55%.

Comparative embodiment 3

Operations in this embodiment were the same as Embodiment 1, and a difference between this embodiment and Embodiment 1 only lied in that, the product prepared in step (1) was not treated in step (2), and was directly collected in a cold trap absorption apparatus using liquid nitrogen as cooling liquid. Results showed that, 607.3 g of crude hexafluoro-1,3-butadiene was collected in a cold trap 1, with purity being 41.7%, converted to 253.4 g on a hundredweight basis, a theoretical yield was 324.0 g, and a yield was 78.2%.

1. A method for preparing hexafluoro-1,3-butadiene, comprising the following steps:
step (1), placing a trifluoroethenyl zinc bromide solution, a catalyst, and a first organic solvent into a pressure resistant reactor, adding bromotrifluoroethylene after displacement with an inert gas, and obtaining hexafluoro-1,3-butadiene through reaction, wherein
the catalyst comprises a monovalent copper salt or a monovalent copper oxide and an organophosphine ligand;
the organophosphine ligand is selected from any one or more of 1,10-phenanthroline, PPh₃, PR₁R₂R₃, PPhR₁R₂, PPh₂R₁, 1,2-bis(diphenylphosphino)ethane, and 2,2′-bis(diphenylphosphino) biphenyl, wherein R₁, R₂, and R₃ are each independently selected from any one of n-propyl, isopropyl, n-butyl, isobutyl, and tert-butyl;
the first organic solvent uses a polar aprotic organic solvent;
step (2), after the reaction is completed, allowing a gas-phase material to pass through a circulating spray system filled with a second organic solvent to absorb excess bromotrifluoroethylene and fluoro-olefin impurities generated during reaction, and purifying the unabsorbed crude hexafluoro-1,3-butadiene through distillation, wherein
the second organic solvent is selected from the polar aprotic organic solvent or ionic liquid.

2. The method for preparing hexafluoro-1,3-butadiene according to claim 1, wherein the monovalent copper salt is selected from any one or more of cuprous iodide, cuprous bromide, cuprous chloride, and cuprous acetate.

3. The method for preparing hexafluoro-1,3-butadiene according to claim 1, wherein the first organic solvent is selected from any one or more of N,N-dimethylformamide, N,N-dimethylacetamide, hexamethylphosphoramide, dimethyl sulfoxide, sulfolane, and N-methyl-2-pyrrolidone, and a moisture content of the polar aprotic organic solvent used in the first organic solvent is ≤500 ppm.

4. The method for preparing hexafluoro-1,3-butadiene according to claim 3, wherein the first organic solvent in step (1) is selected from one of the N,N-dimethylformamide, the N,N-dimethylacetamide, and the sulfolane.

5. The method for preparing hexafluoro-1,3-butadiene according to claim 1, wherein a mole ratio of trifluoroethenyl zinc bromide in the trifluoroethenyl zinc bromide solution to the monovalent copper salt or monovalent copper oxide is 1:(0.01-0.3); and a mole ratio of the monovalent copper salt or monovalent copper oxide to the organophosphine ligand is 1:(1.0-10).

6. The method for preparing hexafluoro-1,3-butadiene according to claim 5, wherein the mole ratio of the trifluoroethenyl zinc bromide in the trifluoroethenyl zinc bromide solution to the monovalent copper salt or monovalent copper oxide is 1:(0.01-0.20).

7. The method for preparing hexafluoro-1,3-butadiene according to claim 1, wherein a mole ratio of the trifluoroethenyl zinc bromide in the trifluoroethenyl zinc bromide solution to the bromotrifluoroethylene is 1:(1-10).

8. The method for preparing hexafluoro-1,3-butadiene according to claim 1, wherein a reaction temperature in step (1) is 60-140° C., a reaction pressure is 0.05-2.0 MPa, and a reaction time is 3-24 h.

9. The method for preparing hexafluoro-1,3-butadiene according to claim 1, wherein the second organic solvent in step (2) is selected from any one or more of the N,N-dimethylformamide, the N,N-dimethylacetamide, the sulfolane, the 1-butyl-3-methylimidazole phosphate, the 1-butyl-3-methylimidazolium acetate, the 1-butyl-3-methylimidazolium trifluoroacetate, and the 1-butyl-3-methylimidazolium hexafluorophosphate.

10. The method for preparing hexafluoro-1,3-butadiene according to claim 1, wherein the circulating spray system in step (2) is a two-stage circulating spray absorption system, and a temperature in the circulating spray system is-10 to 20° C., and a pressure is 0-0.5 MPa.

11. The method for preparing hexafluoro-1,3-butadiene according to claim 1, wherein an absorbed organic solution in the circulating spray system in step (2) is treated through distillation, and the bromotrifluoroethylene is recycled and directly circulated for use.

12. The method for preparing hexafluoro-1,3-butadiene according to claim 1, wherein a content of the bromotrifluoroethylene in the crude hexafluoro-1,3-butadiene treated by the circulating spray system is ≤0.3%, a content of heptafluorobutene is ≤0.02%, and a hexafluoro-1,3-butadiene product with purity ≥99.99% is obtained after distillation.

13. The method for preparing hexafluoro-1,3-butadiene according to claim 1, wherein the monovalent copper oxide is cuprous oxide.

14. The method for preparing hexafluoro-1,3-butadiene according to claim 1, wherein the polar aprotic organic solvent used in the second organic solvent is selected from any one or more of the N,N-dimethylformamide, the N,N-dimethylacetamide, the hexamethylphosphoramide, the dimethyl sulfoxide, and the sulfolane; the ionic liquid is selected from any one or more of 1-butyl-3-methylimidazole phosphate, 1-butyl-3-methylimidazolium methanesulfonate, 1-butyl-3-methylimidazolium acetate, 1-butyl-3-methylimidazolium trifluoroacetate, 1-butyl-3-methylimidazolium nitrate, 1-butyl-3-methylimidazolium (L)-lactate, and 1-butyl-3-methylimidazolium hexafluorophosphate.

15. The method for preparing hexafluoro-1,3-butadiene according to claim 6, wherein the mole ratio of the trifluoroethenyl zinc bromide in the trifluoroethenyl zinc bromide solution to the monovalent copper salt or monovalent copper oxide is 1:(0.05-0.10).

16. The method for preparing hexafluoro-1,3-butadiene according to claim 5, wherein the mole ratio of the monovalent copper salt or monovalent copper oxide to the organophosphine ligand is 1:(1.0-5.0).

17. The method for preparing hexafluoro-1,3-butadiene according to claim 16, wherein the mole ratio of the monovalent copper salt or monovalent copper oxide to the organophosphine ligand is 1:(1.0-3.0).

18. The method for preparing hexafluoro-1,3-butadiene according to claim 7, wherein a mole ratio of the trifluoroethenyl zinc bromide in the trifluoroethenyl zinc bromide solution to the bromotrifluoroethylene is 1:(1.0-5.0).

19. The method for preparing hexafluoro-1,3-butadiene according to claim 8, wherein the reaction temperature in step (1) is 80-120° C., the reaction pressure is 0.3-1.0 MPa, and the reaction time is 6-12 h.

20. The method for preparing hexafluoro-1,3-butadiene according to claim 10, wherein the temperature in the circulating spray system is-5 to 10° C., and the pressure is 0-0.2 MPa.
